(19) United States
(12) Patent Application Publication
(10) Pub. No.: US 2008/0032940 A1
(43) Pub. Date: Feb. 7, 2008

(54) METHODS FOR REDUCING ANTHRACYCLINE-INDUCED TOXICITY

(76) Inventors: Balaraman Kalyanaraman, Wauwatosa, WI (US); Shasi Vardhan Kalivendi, Hyderabad (IN); Joy Joseph, New Berlin, WI (US)

Correspondence Address: QUARLES & BRADY LLP, 33 E. MAIN ST, SUITE 900, P.O BOX 2113, MADISON, WI, 53701-2113, US

(21) Appl. No.: 11/834,799

(22) Filed: Aug. 7, 2007

Related U.S. Application Data
(60) Provisional application No. 60/836,247, filed on Aug. 7, 2006.

(51) Int. Cl. A61K 31/66; A61K 31/704; A61P 35/00; A61P 35/02

(52) U.S. Cl. 514/34; 514/130

(57) ABSTRACT

Methods for treating cancers/tumors, including administering to a subject an effective amount of a mitochondria-targeted antioxidant alone or in combination with a chemotherapeutic agents. Likewise, methods for mitigating toxicity associated with a chemotherapeutic agent including administering an effective amount of a mitochondria-targeted antioxidant with a single or with multiple chemotherapeutic agents.

### CROSS-REFERENCE TO RELATED APPLICATIONS

This application claims the benefit of U.S. Provisional Patent Application No. 60/836,247, filed Aug. 7, 2006, incorporated herein by reference as if set forth in its entirety.

### STATEMENT REGARDING FEDERALLY SPONSORED RESEARCH OR DEVELOPMENT

This invention was made with United States government support awarded by the following agency: National Institute of Cancer, CA77822. The United States has certain nights in this invention.

### BACKGROUND

The invention relates generally to methods for attenuating chemotherapeutic-induced toxicity in normal cells while simultaneously enhancing chemotherapeutic-induced toxicity in tumor cells, and more particularly to co-administering a mitochondria-targeted antioxidant with a chemotherapeutic agent to attenuate the agent's toxicity to normal cells and to enhance its toxicity to tumor cells.

Anthracyclines are cytotoxic antibiotics that are used as chemotherapeutic agents in a wide range of cancers. Anthracyclines include, but are not limited to, doxorubicin (DOX), daunorubicin, epirubicin, idarubicin and mitoxantrone. These agents' cytotoxicity has been attributed to the following: (1) intercalation iii DNA and RNA; (2) generation of free radicals; (3) binding and alkylating DNA; (4) cross-linking DNA; (5) interfering with DNA unwinding, with DNA strand separation and with helicase activity; (6) lipid oxidation of membranes; and (7) inhibition of topoisomerase II. Peng X, et al., “The cardiotoxicology of anthracycline chemotherapeutics: translating molecular mechanism into preventative medicine,” Mol. Interv. 5:163-171 (2005); and Kotamraju S, et al., “Transferrin receptor-dependent iron uptake is responsible for doxorubicin-mediated apoptosis in endothelial cells: role of oxidant-induced iron signaling in apoptosis,” J. Biol. Chem. 277:17179-17187 (2002). A major drawback to anthracyclines is that they damage not only tumor cells, but also normal cells, especially endothelial cells and cardiomyocytes. Kotamraju S, et al., “Doxorubicin-induced apoptosis in endothelial cells and cardiomyocytes is ameliorated by nitrone spin traps and ebselen. Role of reactive oxygen and nitrogen species,” J. Biol. Chem. 275:33585-33592 (2000); and Sawyer D, el al., “Daunorubicin-induced apoptosis in rat cardiac myocytes is inhibited by dexrazoxane,” Circ. Res. 84:257-265 (1999).

Of particular interest herein is DOX-induced toxicity. DOX is a quinione-containing anthracycline used to treat a wide variety of cancers including breast cancer, prostate cancer, Hodgkin's disease and leukemia. DOX is associated with an increased risk of cardiomyopathy or congestive heart failure, especially when used to treat leukemia in children. In fact, children who undergo DOX chemotherapy are nearly eight times more likely to develop heart problems later in life. Wang S, et al., “Doxorubicin induces apoptosis in normal and tumor cells via distinctly different mechanisms. Intermediacy of H₂O₂— and p53-dependent pathways,” J. Biol. Chem. 279:25535-25543 (2004), incorporated herein by reference as if set forth in its entirety.

Wang et al. demonstrated that DOX initiates apoptosis via a different mechanism in normal cells than it does in tumor cells. In normal cells (e.g., endothelial cells and cardiomyocytes), DOX generates reactive oxygen species (ROS), such as hydrogen peroxide (H₂O₂) and (superoxide (O₂⁻), to cause apoptosis. Wang et al., supra. In addition, DOX-induced ROS alter intracellular calcium homeostasis in normal cells, contributing to DOX's toxicity. Kalivendi S, et al., “Doxorubicin activates nuclear factor of activated T-lymphocytes and Fas ligand transcription: role of mitochondrial reactive oxygen species and calcium,” Biochem. J. 389:527-539 (2005). In contrast, in tumor cells, DOX causes early activation of p53 tumor suppressor to cause apoptosis. Wang et al., supra. Accordingly, these different mechanisms of apoptosis provide a potential strategy for minimizing DOX-induced toxicity without attenuating its anti-tumor potential.

Recently, researchers proposed that antioxidants may help to attenuate DOX-induced toxicity in normal cells, and in particular, cardiomyocytes. For example, coenzyme Q (CoQ) reversed DOX-induced toxicity in isolated cardiomyocytes and in isolated hearts. Conklin K, “Coenzyme Q₁₀ for prevention of anthracycline-induced cardiotoxicity,” Integr. Cancer Ther. 4:110-130 (2005), incorporated herein by reference as if set forth in its entirety. However, it was not as effective in animal models. Id. Likewise, α-tocopherol and N-acetylcysteine showed limited efficacy in preventing cardiotoxicity in laboratory animals. Berthiaume J, et al., “Dietary vitamin E decreases doxorubicin-induced oxidative stress without preventing mitochondrial dysfunction,” Cardiovasc. Toxicol. 5:257-267 (2005); and Doroshow J, et al., “Prevention of doxorubicin cardiac toxicity in the mouse by N-acetylcysteine,” J. Clin. Invest. 68:1053-1064 (1981). The limited efficacy of these antioxidants is most likely because they have low solubility in water and because they do not readily translocate into mitochondria, which are particularly sensitive to DOX-induced toxicity. Conklin, supra. In addition, these antioxidants protect not only normal cells, but also tumor cells in DOX-treated subjects, thereby reducing DOX's overall effectiveness. Iarussi D, et al., “Anthracycline-induced cardiotoxicity in children with cancer: strategies for prevention and management,” Paeditr. Drugs 7:67-76 (2005).

To overcome these drawbacks, Kelso et al. synthesized an antioxidant that is a mixture of mitoquinone (10-(6′-ubiquinonyl)decyltriphenylplhosphonium bromide; oxidized form) and mitoquinol (10-(6′-ubiquinolyl)decyltriphenylphosplhonium bromide; reduced form), collectively called MitoQ, that specifically targets mitochondria. Kelso G, et al., “Selective targeting of a redox-active ubiquinone to mitochondria within cells: antioxidant and antiapoptotic properties,” J. Biol. Chem. 276:4588-4596 (2001), incorporated herein by reference as if set forth in its entirety. As shown in FIG. 1A-1B, MitoQ is a ubiquinone/ubiquinol derivative comprising CoQ (FIG. 1C) conjugated to a lipophilic triphienylphosphonium cation through an aliphatic carbon chain. James A, et al., “Interactions of mitochondria-targeted and untargeted ubiquinones with the mitochondrial respiratory chain and reactive oxygen species. Implications for the use of exogenous ubiquinones as therapies and experimental tools,” J. Biol. Chem. 280:21295-21312 (2005); and Smith R, et al., “Delivery of bioactive molecules to mitochondria in vivo,” Proc. Natl. Acad. Sci. USA 100:5407-5412 (2003), each of which is incorporated herein by reference as if set forth in its entirety.

MitoQ accumulates fifty to one-hundred fold in mitochondria when compared to untargeted antioxidants. The lipophilic triphenylphosphonium cation easily permeates through lipid bilayers and subsequently accumulates in mitochondria because of its large membrane potential. In addition, MitoQ acts as a potent antioxidant in endothelial cells by inhibiting peroxide-induced oxidative damage and apoptosis. Dhanasekaran A, et al., “Supplementation of endothelial cells with mitochondria-targeted antioxidants inhibit peroxide-induced mitochondrial iron uptake, oxidative damage, and apoptosis,” J. Biol. Chem. 279:37575-37587 (2004); see also Adlam V, et al., “Targeting an antioxidant to mitochondria decreases cardiac ischemia-reperfusion injury,” FASEB J. 19:1088-1095 (2005). Furthermore, MitoQ protects central nervous system cells from oxidative damage. Jauslin M, et al., “Mitochondria-targeted antioxidants protect Friedreich Ataxia fibroblasts from endogenous oxidative stress more effectively than untargeted antioxidants,” FASEB J. 17:1972-1974 (2003). The effects of mitochondria-targeted antioxidants on tumor cells, however, have not yet been established.

For the foregoing reasons, there is a need for methods of attenuating the toxic effect of anthracyclines on normal cells, while simultaneously enhancing their effect on tumor cells.

### BRIEF DESCRIPTION OF THE DRAWINGS

The invention will be better understood and features, aspects and advantages other than those set forth herein will become apparent when consideration is given to the following detailed description thereof. Such detailed description makes reference to the following drawings, wherein:

FIGS. 1A-1C show the structures of the mitoquinone (FIG. 1A) and the mitoquinol (FIG. 1B) forms of MitoQ, as well as the parent, CoQ (FIG. 1C). “Me” indicates a methyl group, “Br” indicates a bromide and “Ph” indicates a phenyl group;

FIG. 2 shows the dose-dependent, differential toxicity of MitoQ in two normal cell lines (bovine aortic endothelial cells (BAEC) and H9c2) compared to three tumor cell lines (SH-SY5Y, MCF-7 and LnCAP) as measured by a MTT assay;

FIG. 3 shows morphology by phase-contrast microscopy of normal cells and tumor cells in response to low concentrations of MitoQ;

FIG. 4 shows the differential effect of MitoQ on tumor cells and normal cells, as measured by a MTT assay. For each cell line tested, the first bar is control, the second bar is MitoQ, the third bar is TPP and the fourth bar is CoQ;

FIG. 5 shows the differential effect of MitoQ on DOX-induced caspase-3 activity tumor cells and normal cells; and

FIG. 6 shows the differential effect of MitoQ and DOX on nuclear morphology in tumor cells and normal cells;

FIGS. 7A-C show cell viability in response to DOX, MitoQ or CoQ at twenty-four and forty-eight hours in tumor cells and normal cells;

FIG. 8 shows a timeline of treatment as described in Example 4.

FIG. 9A shows echocardiograms of control and DOX-treated rat hearts following treatment as described in Example 4. As shown, MitoQ, but not CoQ, mitigates DOX-induced cardiomyopathy after 12 weeks of DOX treatment. FIG. 9B shows global radial strain in each of the treatment groups.

FIG. 10 shows cyctochrome c oxidase activity in each of the treatment groups in Example 4.

FIGS. 11A-B show the effects of DOX and Mito-Q on myocardial fibrosis.

FIG. 12 shows the effects of DOX and Mito-Q on caspase 3 activity.

FIG. 13 shows a timeline of treatment as described in Example 5.

### DESCRIPTION OF PREFERRED EMBODIMENTS

The present invention relates to our observation that at low micromolar concentrations, mitochondria-targeted antioxidants, like MitoQ, differentially affect normal cells and tumor cells. This observation suggests that mitochondria-targeted antioxidants may synergize with a chemotherapeutic agent, like DOX, enhancing the anti-tumor potential of the chemotherapeutic agent, while simultaneously attenuating its toxic side-effects to normal cells, especially cardiomyocytes. As such, one can selectively induce apoptosis in tumor cells while protecting normal cells, thereby increasing the effectiveness of the chemotherapeutic agent.

Unless defined otherwise, all technical and scientific terms used herein have the same meaning as commonly understood by one of ordinary skill in the art to which the invention belongs. Although any methods and materials similar to or equivalent to those described herein can be used in the practice or testing of the present invention, the preferred methods and materials are now described.

As used herein, an anthracycline means an agent having a characteristic four ring structure linked via a glycosidic bond to daunosamine. Examples of anthracyclines include, but are not limited to DOX, daunorubicin, epirubicin, idarubicin and mitoxantrone.

As used herein, anthracycline-induced cardiotoxicity refers to damage that occurs to cardiomyocytes following anthracycline administration. In particular, it refers to EKG changes and arrhythmias, or cardiomyopathy leading to congestive heart failure that occurs in subjects administered anthracyclines.

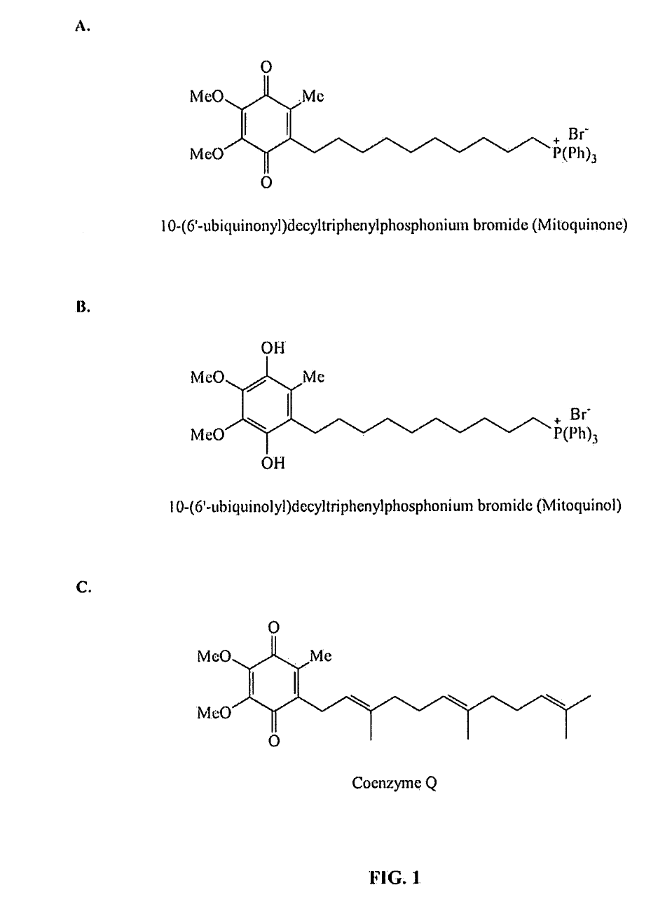

As used herein, MitoQ refers to a mixture of the compounds shown in FIGS. 1A and 1B or to each compound individually. It is contemplated that any antioxidant having a CoQ moiety modified for targeting to mitochondria can be used herein. Example of such mitochondria-targeted antioxidants include, but are not limited to, Mito-TEMPOL, and MitoE (vitamin E attached to a CoQ).

The Examples below use DOX; the invention, however, is not intended to be limited to this particular anthracycline. It is contemplated that the methods described below may be used with any anthracycline that causes cardiotoxicity.

In one embodiment, the present invention is the use of a mitochondria-targeted antioxidant, preferably MitoQ, in the treatment of cancer, including tumors. Although previously known to protect normal cells from oxidative stress, it is surprisingly shown below that MitoQ is paradoxically toxic to cancer cells. As such, it is envisioned that MitoQ may be administered alone to selectively induce apoptosis in cancer cells. It is also envisioned that because children are more susceptible to anthracycline-induced cardiotoxicity, MitoQ alone may be an effective for treatment for certain childhood cancers, thereby reducing the use of anthracyclines to treat tumors in children. It is further envisioned that MitoQ may be used alone in adults as a preventative measure in adults susceptible to certain types of cancers or that MitoQ may be used alone to treat low-grade tumors.

This embodiment envisions selecting a subject having or susceptible to having a cancer and administering an effective amount of MitoQ so that the cancer is reduced by at least about 10%, preferably by at least about 15%, more preferably by at least about 20%, and most preferably by at least about 25% (e.g., tumor size/mass or cell number is reduced). MitoQ is best administered as an intraperitoneal solution, although intravenous and oral forms are contemplated. MitoQ is stable in water, as well as saline solutions at a pH of about 7.0. MitoQ is sensitive to light and must therefore be properly protected to prevent degradation. Pharmaceutically acceptable Carriers for MitoQ include MS-010 (MitoQ mesylate:beta cyclodextrin adduct; a 20-25% w/w MitoQ complex with beta-cyclodextrin; Douglas Pharmaceuticals, Ltd.; Auckland, New Zealand)

Dosages suitable for this embodiment include 40 to 80 mg of MitoQ at least three times a week. However, the dosage and treatment with MitoQ will differ for different subjects. Likewise, the number of doses a subject receives, the time allowed between doses and the length of time a subject receives MitoQ will generally depend on the severity of the cancer.

For example, in a preferred embodiment, MitoQ may be given to a subject intravenously stating at least about 5 to 10 mg/kg three times a week. This dosage level and the time between doses may be modified based on a physician's assessment of the disease progression.

In another embodiment, the present invention is the use of a mitochondria-targeted antioxidant, preferably MitoQ, in combination with an anthracycline in the treatment of certain cancers. In this embodiment, it is envisioned that MitoQ will not only protect normal cells from the oxidative stress that ensues from anthracycline administration, but also induce apoptosis in tumor cells. Accordingly, MitoQ reduces anthracycline-induced cardiotoxicity by two mechanisms. The first mechanism is by protecting cardiomyocytes from oxidative stress. The second mechanism is by synergizing with the anthracycline, thereby reducing the amount of anthracycline that is required to be administered.

This embodiment envisions selecting a subject having or susceptible to having a cancer and co-administering an effective amount of MitoQ and a effective amount of an anthracycline, particularly DOX, so that the cancer is reduced by at least about 10%, preferably by at least about 15%, more preferably by at least about 20%, and most preferably by at least about 25%. The combination of MitoQ and the anthracycline is best administered as an intraperitoneal solution, although both can be given intravenously and orally. The combination is stable in water, as well as saline solutions at a pH of about 7.0. As noted above, MitoQ is sensitive to light and must therefore be protected to prevent degradation. Pharmaceutically acceptable carriers the combination include MS-010.

Dosages suitable for this embodiment include least about 40 to 80 mg of MitoQ and 100 to 200 mg/m² of DOX three times a week. However, the dosage and treatment with MitoQ and DOX will differ for different subjects likewise, the number of doses a subject receives, the time allowed between doses and the length of time a subject receives MitoQ and DOX will generally depend on the severity of the cancer. Because of DOX's toxicity, bone marrow toxicity measurements should be made periodically. If toxicity becomes apparent, the dosage should be reduced to twice a week.

For example, in a preferred embodiment, MitoQ may be given to a subject in an injection dosage form starting at least about 5 to 10 mg/kg of MitoQ three times a week intravenously with 100 to 200 mg/m² DOX. Isner J, et al., “Clinical and morphologic cardiac findings after anthracycline chemotherapy. Analysis of 64 patients studied al necropsy,” Am. J. Cardiol. 51:1167-1174 (1983). In preferred embodiments, MitoQ is administered not only with DOX, but also before and after DOX because of its cumulative toxicity. This dosage level and the time between doses may be modified based on a clinician's assessment of the disease progression.

The invention will be more fully understood upon consideration of the following non-limiting Examples.

### EXAMPLES

### Example 1

### MitoQ Synthesis

Methods: MitoQ was synthesized according to a method described by Kelso et al., supra. Briefly, 11-bromoperoxyundecanoic acid, prepared from 11-bromoundecanoic acid, was coupled with 2,3,-dimethoxy-5-methyl-1,4-benzoquinone to yield 6-(10-bromodecyl) ubiquinone. The quinone was reduced to the quinol using sodium borohydride and heated with triphenylphosphine in dioxane for four days. An oily product separated from the reaction medium was purified and was analyzed by mass spectroscopy.

Matsunaga et al. have previously shown that MitoQ is non-toxic to BAECs. Matsunaga T, et al., “Ceramide-induced intracellular oxidant formation, iron signaling, and apoptosis in endothelial cells: protective role of endogenous nitric oxide,” J. Biol. Chem. 279:28614-28624 (2004). A similar result was reported by Dhanasekaranl et al. in an embryonic cardiomyocyte cell line, H-9c2. Dhanasekaran A, et al., “Supplementation of endothelial cells with mitochondria-targeted antioxidants inhibit peroxide-induced mitochondrial iron uptake, oxidative damage, and apoptosis,” J. Biol. Chem. 279:37575-37587 (2004). The H9c2 cell line is a frequently used cell line that mimics primary myocytes. However, the effect of MitoQ on cancer/tumor cells has yet to be reported.

### Example 2

### Differential Effect of Mitochondria-Targeted Antioxidants on Normal Cells and Tumor Cells

Methods: MitoQ was produced as described above.

Cell lines: Two normal cell lines were used herein. The first normal cell line was a rat-derived embryonic cardiomyocyte cell line, H9c2, from American Tissue Type Collection (Manassas, Va.; Catalog No. CRL-1446). The second normal cell line was BAECs from Clonetics Corp. (San Diego, Calif.).

In contrast, four tumor cell lines were used herein. The first tumor cell line was a human neuroblastoma cell line, SH-SY5Y, from American Type Culture Collection (ATCC; Rockville, Md.). The second tumor cell line was a human prostate cancer cell line, LnCAP, from ATCC. The third tumor cell line was a human breast cancer cell line, MCF-7, from ATCC. The fourth tumor cell line was a human metastatic breast cancer cell line, MDA-MB-231 from ATCC.

In a first set of experiments, a dose-response curve to MitoQ was measured in each of the six cell lines described above. Briefly, cells were treated with various concentrations of MitoQ for twenty-four hours. Following treatment, cellular toxicity was measured by a MTT assay as described below.

In a second set of experiments, MCF-7, SH-SY5Y, H9c2 and LnCAP cell lines were subjected to the following three treatments: (1) control; (2) 10 μM MitoQ for twenty-four hours; and (3) 4 μM MitoQ for twenty-four hours. Following treatment, cellular morphology was examined by phase-contrast microscopy as described below.

In a third set of experiments, each of the six cell lines described above were subjected to the following treatments: (1) 10 μM MitoQ; (2) 10 μM CoQ (MitoQ without the phosphonium, which served as a control); and (3) 10 μM triphenylphosphonium (TPP; MitoQ without the ubiquinone or the ubquinol moiety, which also served as a control). Following treatment, cellular toxicity was measured by the MTT assay.

MTT Assay: An MIT assay was performed to examine MitoQ's toxicity on normal cells and tumor cells (see Mosmann T, “Rapid colorimetric assay for cellular growth and survival: Application to proliferation and cytotoxicity assays,” J. Immunol. Meth. 65:55-63 (1983)). Briefly, cellular toxicity was measured by the addition of 0.5% MTT dye (3-(4,5-Dimethlthiazol-2-yl)-2,5-diphenyltetrazolium bromide; Promega; Madison, Wis.) following treatment. MTT-treated cells were then incubated at 37° C. for 2 hours. Formazon crystals formed cells were solubilized by adding 200 μl of 0.1N HCl in isopropanol, and absorbance was measured at 570 nm.

Phase-Contrast Morphology: Cellular morphology % vas performed to assess toxicity of MitoQ. Cell were examined using a Nikon Diaphot inverted microscope with an Odyssey confocal imaging system (Noran Instruments; Middleton, Wis.).

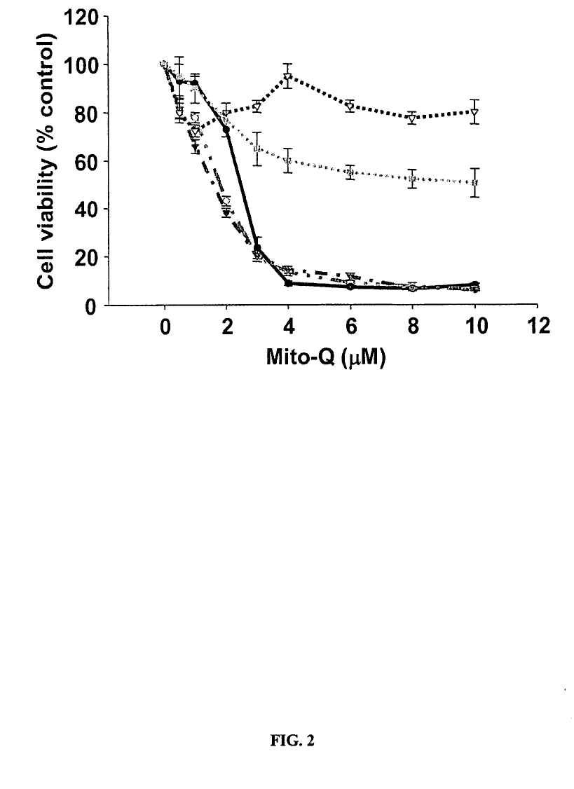

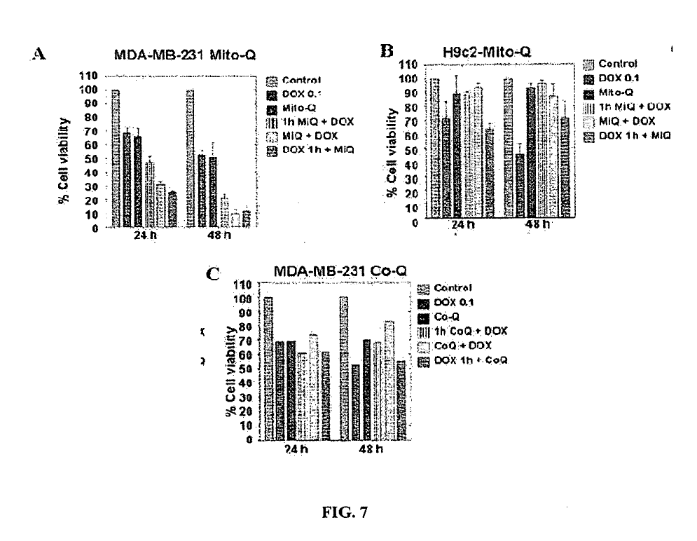

Results: As shown in FIG. 2, tumor cell lines (i.e., LnCAP, MCF-7, SH-SY5Y and MDA-MB-231 (FIG. 7)) demonstrated a dose-dependent toxicity to MitoQ that was not observed in normal cells (i.e., H9c2 and BAECs). That is, BAECs and H9c2 cells showed greater than 50% cell viability for all levels of MitoQ test. In general, the LD₅₀ levels of MitoQ in tumor cells were about 1-2 μM; whereas, the LD₅₀ levels of MitoQ in normal cells were at least 8-10 fold higher.

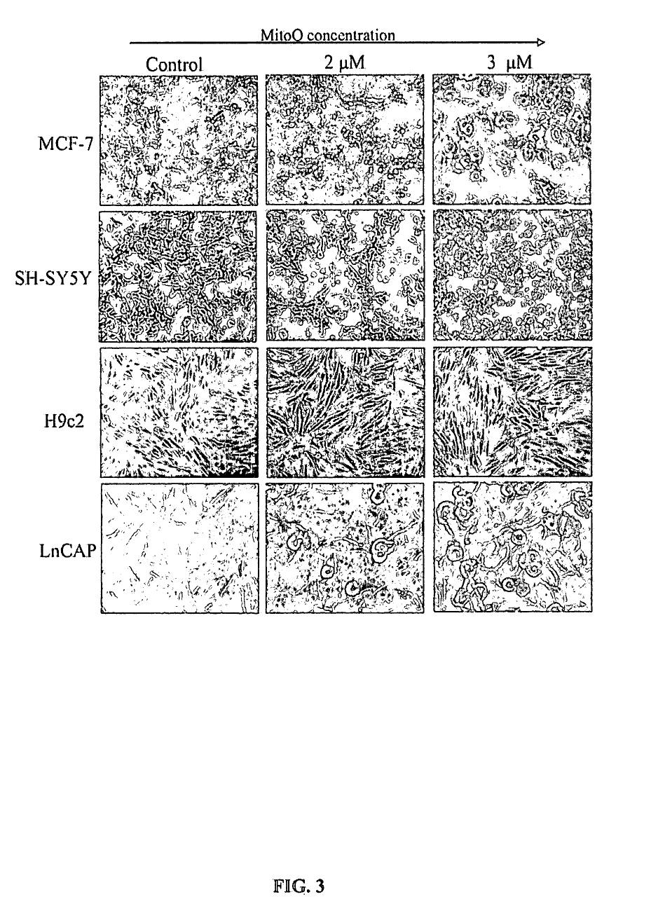

As shown in FIG. 3, low concentrations of MitoQ were more toxic to tumor cell lines (MCIF-7, SH-SY5Y and LNCAP) than to normal cells (H9c2). At low concentrations, MitoQ caused shrinking, blebbing and nuclear condensation of MCF-7 cells and other cancer cells. However, MitoQ did not cause similar effects in normal cells.

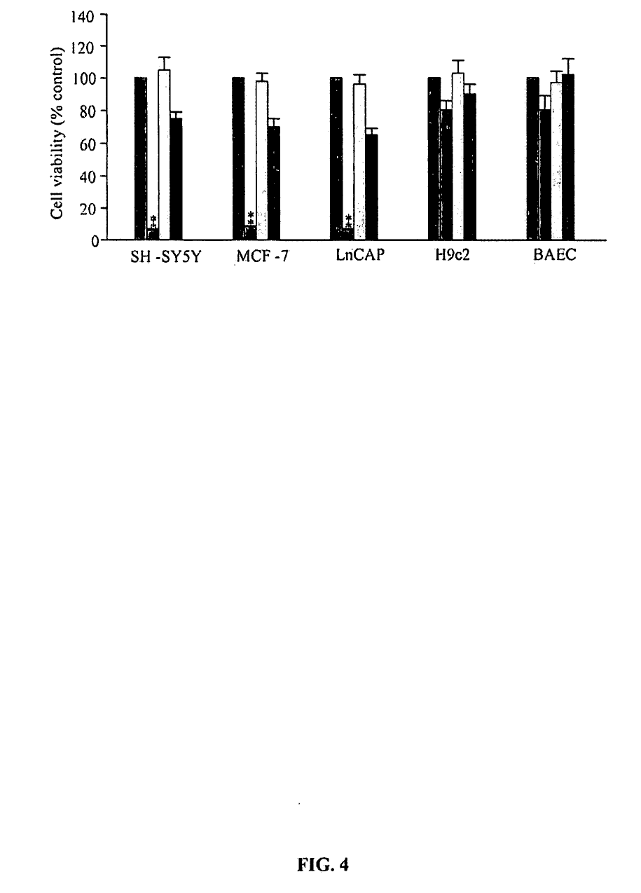

As shown in FIG. 4, MitoQ was selectively toxic to tumor cells (LnCAP, MCF-7 and SH-SY5Y), but not normal cells (H9c2 and BAECs). Conversely, TPP and COQ were not toxic to any of the cell lines tested. This data indicates that it is MitoQ and not the phosphonium moiety (TPP) or the antioxidant property (CoQ) that causes the selective toxicity to tumor cells.

### Example 3

### Differential Effect of Mitochondria-Targeted Antioxidants on Normal Cells and Tumor Cells During Anthracycline Administration

Methods: MitoQ was produced as described above.

Cell lines: Two normal cell lines were used herein. The first normal cell line was the rat heart-derived embryonic cardiomyocyte cell line, H9c2 cells, described above. The second normal cell line was cardiomyocytes (CM). Adult rat ventricular CMs were isolated from male Sprague-Dawley rats (175-225 g body weight) as previously described. Konorev E, et al., “Bicarbonate exacerbates oxidative injury induced by antitumor antibiotic doxorubicin in cardiomyocytes,” Am. J. Physiol. Circ. Physiol. 279:H2424-H2430 (2000).

In contrast, three tumor cell lines were used herein. The first tumor cell line was the human neuroblastoma cell line, SH-SY5Y, described above. The second tumor cell line was the human prostate cancer cell line, MCF-7, described above. The third tumor cell line was a human breast cancer cell line, MCF-10A, from ATCC.

In a first set of experiments, each cell line was subjected the following four treatments: (1) control (PBS); (2) 0.5 μM DOX; (3) 2 μM MitoQ for 1 hour followed by 0.5 μM DOX; and (4) 2 μM MitoQ. H9c2 and SH-SY5Y were incubated for 16 hours; whereas, CM, MCF-7 and MCF-10A cells were incubated for 48 hours. Following termination of incubation, cells were washed twice with Dulbecco's phosphate-buffered saline (DPBS) and caspase-3 activity was measured as described below.

In a second set of experiments, BAECs, LnCAP and MCF-7 cell lines were subjected to the four treatments described in the previous paragraph. BAECs were incubated for 16 hours; whereas, LnCAP and MCF-7 cells were incubated for 48 hours. Following the temination of incubation, cells were stained with Hoeschst to examine nuclear morphology as described below.

Caspase-3 Activity: Caspase-3 activity was measured with acetyl Asp-Glu-Val-Asp 7-amido-4-methlylcoumarin (Ac-DEVD-AFC), as described in a kit from Sigma Aldrich (St. Louis, Mo.). Caspase-3 hydrolyzes Ac-DEVD-AFC to release a fluorescent moiety, 7-amino-4-methylcoumarin (AMC). Briefly, treated cells were suspended in 100 μl of lysis buffer and passed through a 24-gauge needle 10 times to ensure complete lysis. The resulting lysate was centrifuged at 4° C. at 10,000 rmp. 50 μl of clear supernatant was used for the activity assay. An increase in fluorescence was considered as an index of caspase activity, suggesting increased apoptosis. The excitation and emission wavelengths of AMC are 360 nm and 460 nm respectively.

Hoechst Staining: After termination of incubation (16 hours for BAEC, and 48 hours LnCAP and MCF-7), 10 μg/ml Hoechst was added to the cells and incubated for 20 minutes. The medium was then aspirated, and the cells were washed twice with DPBS. Cells were fixed with 4% formaldehyde and photographed with a Nikon fluorescence microscope. Excitation and emission wavelengths of 360 nm n and 450 nm were used. Cells demonstrating blue staining with condensed nuclei were considered apoptotic.

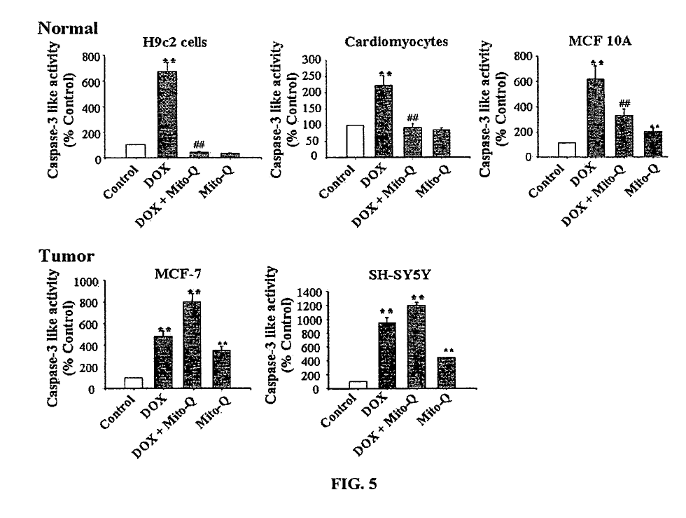

Results: As shown in FIG. 5, MitoQ syngerized with DOX to enhance caspase-3 activity in tumor cell lines (MCF-7, MCF-10A and SH-SY5Y), but not in normal cells lines (CM and 1-19c2). In fact, MitoQ attenuated DOX-induced caspase-3 activity in normal cell lines.

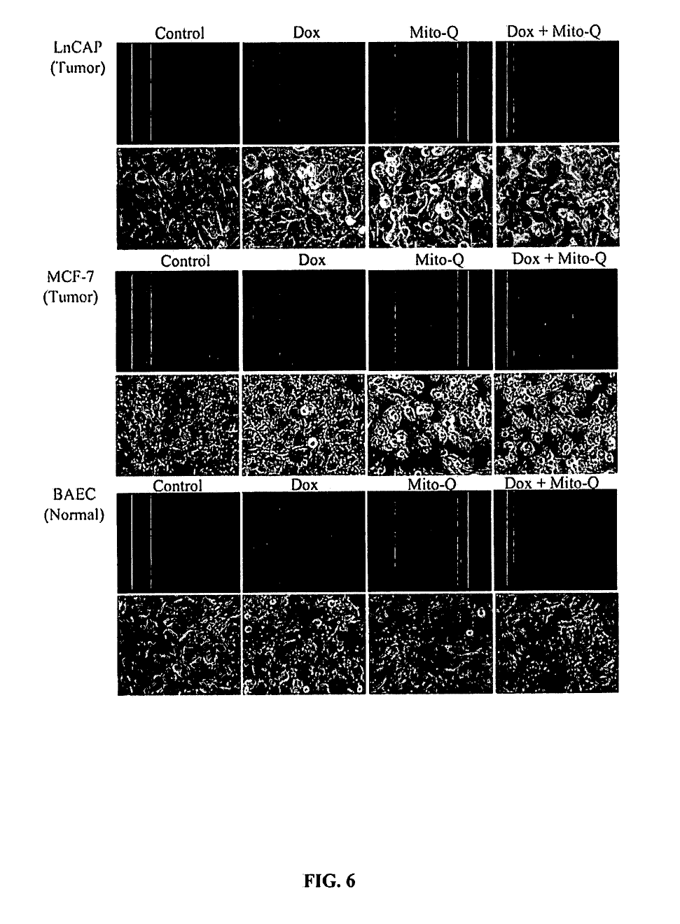

As shown in FIG. 6 MitoQ exacerbated DOX-induced nuclear condensation in the tumor cell lines as compared to the normal cell lines. In fact, MitoQ mitigated the nuclear condensation in the normal cell lines. As shown in FIG. 7, MitoQ synergistically decreased cell viability when MDA-MB-231 breast cancer cells were treated with DOX. Similar effects were not detected with CoQ.

### Example 4

### Cardioprotection via Mitochondria-Targeted Antioxidants During Anthracycline Administration

Methods: MitoQ will be produced as described above.

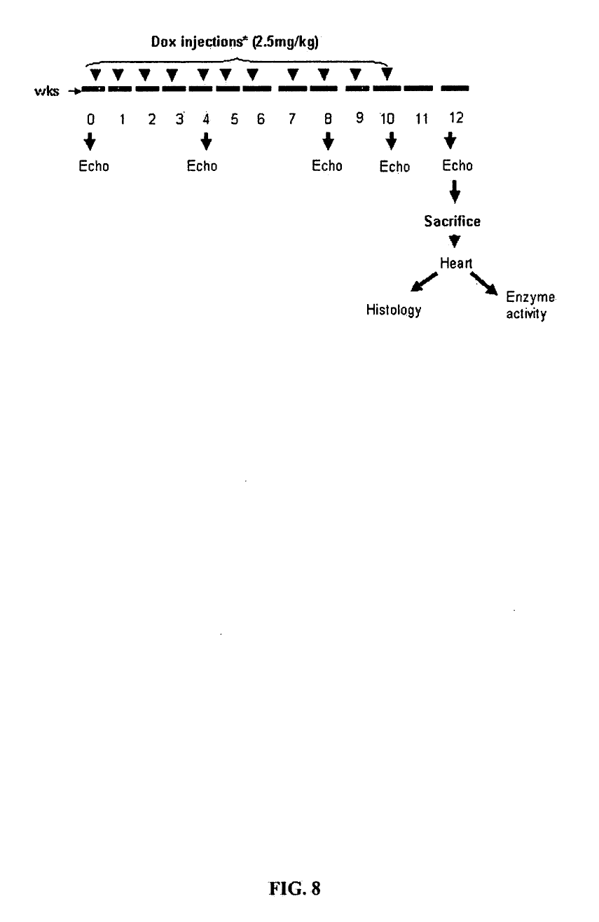

In a first set of experiments, male Sprague Dawley rats were divided into the following four treatment groups: (1) PBS control; (2) MitoQ (5 mg/kg, i.p., twice per week); (3) DOX (2.5 mg/kg, i.v.); and (4) DOX plus MitoQ (2.5 mg/kg DOX and 5 mg/kg MitoQ, both i.p.). All agents were administered in PBS for ten weeks. At weeks four, eight, ten and twelve, cardiac function tests were performed. Following the treatments, the rats were sacrificed and hearts were removed for histochemical and biochemical analysis. See FIG. 8 for treatment protocol

In a second set of experiments, a different mitochondria-target antioxidant was assessed, Mito-TEMPOL. Male Sprague Dawley rats were divided into the following four treatment groups: (1) PBS control; (2) Mito-TEMPOL (5 mg/kg, i.p., twice per week); (3) DOX (2.5 mg/kg, i.v.); and (4) DOX plus Mito-TEMPOL (2.5 mg/kg and 5 mg/kg Mito-TEMPOL, both i.p.). All agents were administered in PBS for ten weeks. At weeks four, eight, ten and twelve, cardiac function tests were performed. Following treatment, the rats were sacrificed and hearts were removed for histochemical and biochemical analysis.

Cardiac Function Test: Echocardiography was performed at baseline, four, eight, ten and twelve weeks following the start of treatment. Rats were anesthetized with a mixture of ketamine (75 mg/kg) and medotomidine (0.25 mg/kg). Once anesthetized, the chest was shaved and cardiac rhythm monitored using three limb leads attached to a Vivid 7 from General Electric (Waukesha, Wis.) and a M12L linear array probe.

2-D B mode imaging parameters were as follows: (1) depth, 2.5 cm.; (2) frame rate, 230 frames/second; and (3) second harmonic imaging. For the primary endpoint data, the standard short axis view at the mid-ventricular level were used. Because of the serial nature of comparative measurements, co-registration at this level was ensured by the use of papillary muscles as localizers and obtaining a circular left ventricular slice. Other standard views obtained induced short axis apical slices and short axis basal slices at the mitral valve. The following data was obtained using of fine M mode analysis from B mode images: (1) left ventricular ejection fraction; (2) fractional shortening; (3) left ventricular internal dimensions in diastole and systole; (4) anterior and inferior wall thickness in diastole and systole; and (4) left ventricular mass. In addition, Q analysis software (General Electric) of the B mode echo images was used to measure regional radial and circumferential strain. Furthermore, diastolic function was monitored by using pulsed Doppler of the mitral inflow and aortic outflow regions in a standard apical four chamber/five chamber view, as was mitral E wave, A wave, isovolumic relaxation time and mitral E deceleration time.

ROS Measurements: ROS was measured in various tissues, including cardiac tissue. ROS was measured by the method of Zliao et al. Zhao H, et al. “Superoxide reacts with hydroethidine but forms a fluorescent product that is distinctly different from ethidium: Potential implications in intracellular fluorescence detection of superoxide,” Free Rad. Biol. Med. 34:1359-1368 (2003), incorporated herein by reference as if set forth in its entirety. Briefly, hydroethidine (HE) reacts with superoxide to Form 2-hydroxyethidium (2-OH-E⁺).

Mitochondrial Function Tests: Mitochondrial complex I and complex IV activity were measured according to the method of Vijayasarathy et al. Vijayasarathy C., et al., “Tissue variant effects or heme inhibitors on the mouse cytochrome c oxidase gene expression and catalytic activity of the enzyme complex,” Eur. J. Biochem. 266:191-200 (1996), incorporated herein by reference as if set forth in its entirety.

Caspase-3 was determined as described above in Example 3.

TUNEL Assay: A terminal deoxynucleotidyl transferase-mediated nick-end labeling assay (TUNEL) was performed as described in an ApoAlert DNA fragmentation assay kit from Clonetech Laboratories (Palo Alto, Calif.). Briefly, apoptotic cells exhibit a strong nuclear green fluorescence that is detected using a standard fluorescein filter (520 nm). Cells were stained with a propidium iodide exhibit a strong red fluorescence at 620 nm. Areas of apoptosis were detected by fluorescence microscopy equipped with rhodamine and fluorescein isothiocyante filters. Images were analyzed with a Metamorph® Imaging System (Universal imaging Corp.; Downingtown, Pa.).

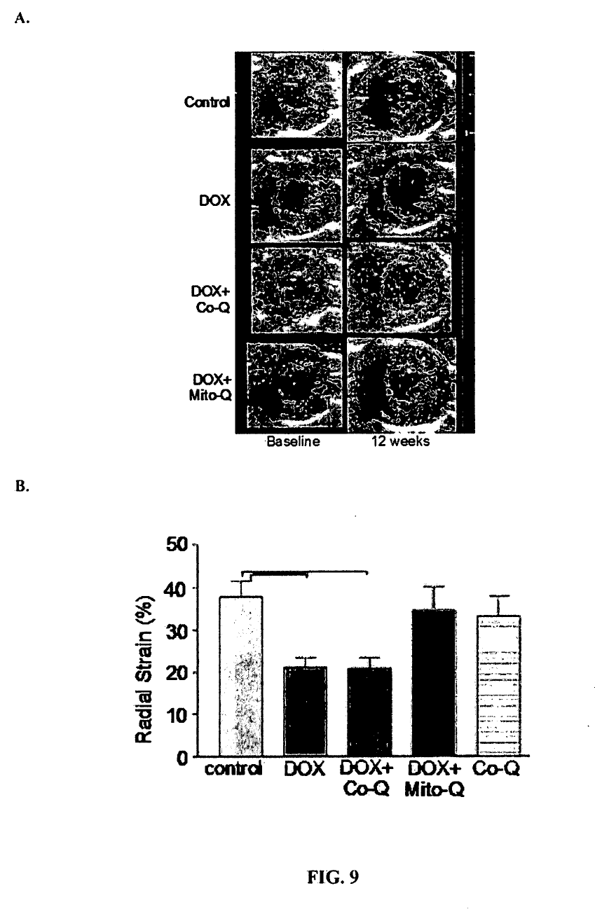

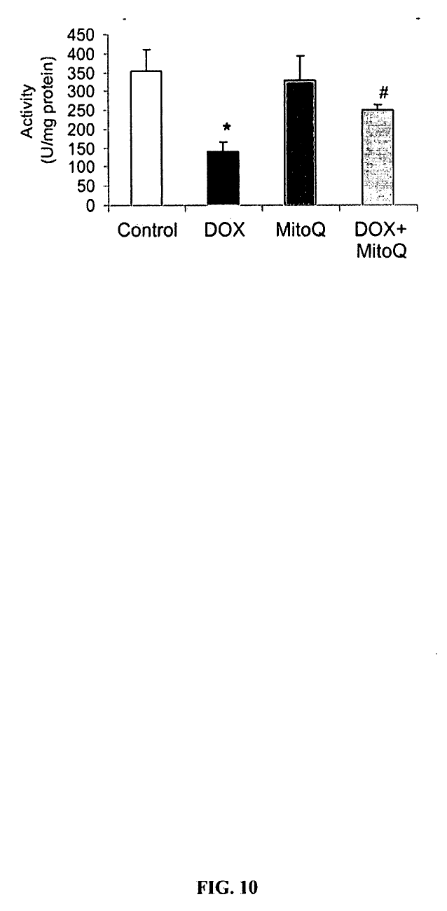

Results: As shown in FIG. 9, DOX treatment significantly decreased fractional shortening (18% versus control) and radial strain (35% versus control) (Migrino, R, et al., in press—Ultrasound in Medicine and Biology). On the other hand, MitoQ administration preserved left ventricular function and fractional shortening as measured by echocardiography. In contrast, CoQ administration did not significantly protect against cardiotoxicity induced by DOX. Alter ten weeks of DOX treatment, mitochondrial fractions were isolated from the heart tissue and both cytochrome c oxidlase activity and mitochondrial complex I activity were measured (FIG. 10).

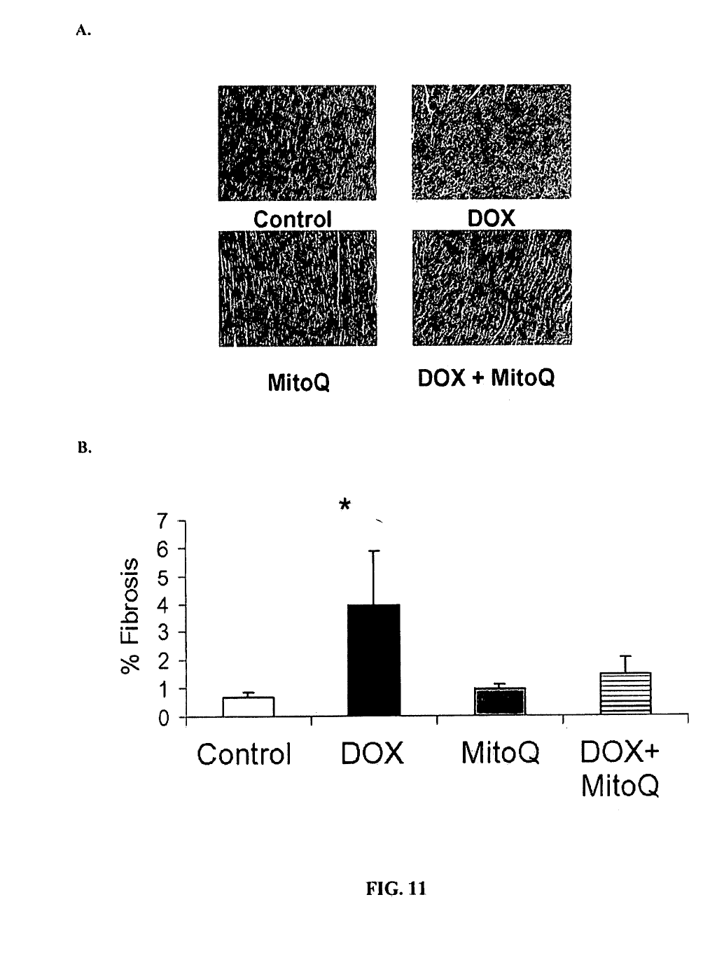

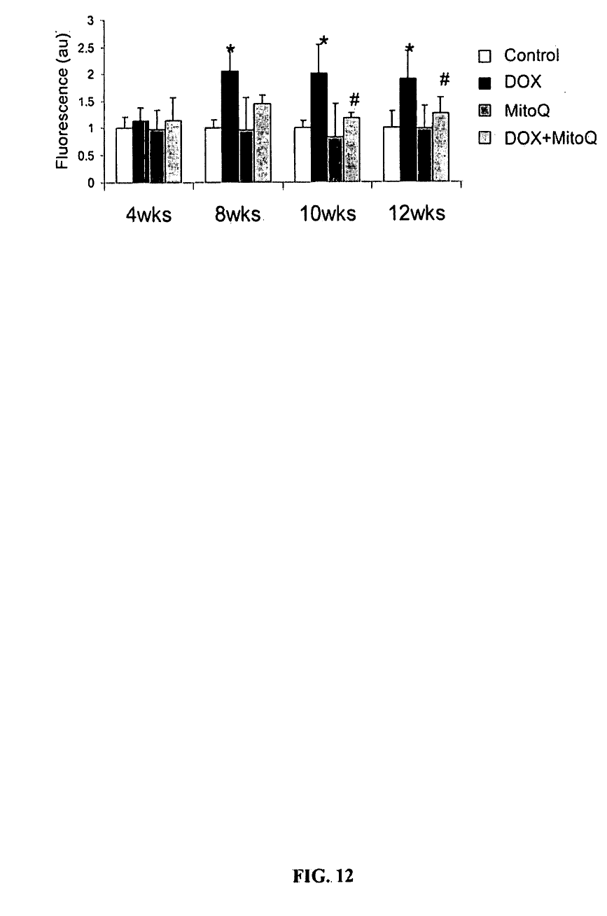

MitoQ administration markedly restored the cytochrome c oxidase activity in hearts isolated from DOX-treated rats. Cardiac tissues isolated from control and DOX-treated rats were stained with Massons Trichrome and the area or fibrosis was measured using Metamorph software. As shown in FIG. 11, the percent fibrosis induced by DOX was mitigated during MitoQ and DOX treatment. As shown in FIG. 12, caspase activity in heart tissues isolated from rats increased with increasing DOX treatment. MitoQ administration markedly inhibited the increase in caspase-3 activity observed during DOX treatment.

Similar results were obtained when Mito-TEMPOL or MitoE (vitamin E linked to CoQ) was administered with DOX (results not shown).
Example 5 (Prophetic)

### Cardioprotection and Synergistic Increase in Antitumor Efficacy During DOX and MitoQ Treatment

Methods: MitoQ will be produced as described above.

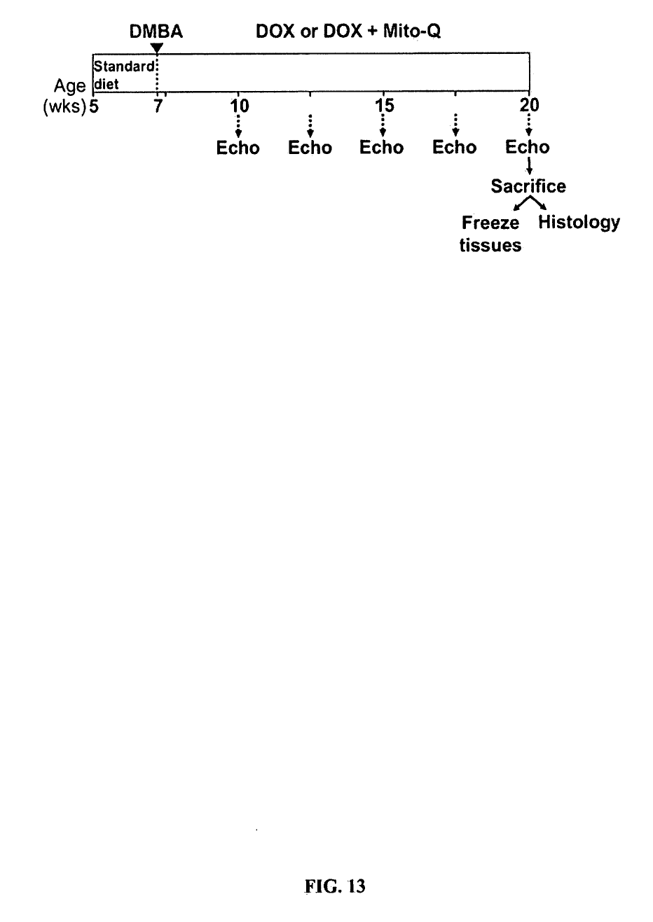

In a first series of experiments, female Sprague-Dawley rats (50-days-old) will receive a single dose of 12-dimethylbenz[a]anthiracene (DMBA) (50 mg/kg body weight) by oral gavage (FIG. 13). Rats will be palpated once a week after three weeks for the presence or mammary tumors. Previous histopathological data shows that >95% of the mammary lesions induced by DMBA within the 120-day time frame are adenocarcinomas. The mean latency to appearance of the first palpable mammary tumor is five to six weeks.

DOX and DOX plus MitoQ administration will begin immediately after the appearance of the first palpable mammary tumor. These experiments will be performed in four groups of six female, 50-day-old Sprague-Dawley rats. In Group 1, rats will be treated with a single oral gavage of DMBA (50 mg/kg). After the appearance of tumor (5-6 weeks), rats will be administered saline alone and rat body weight and (echocardiograms will be obtained periodically over 10-12 weeks. In Group 2, rats will be treated with DMBA, followed by administration of DOX (2.5 mg/kg, IV) once/week for 10-12 weeks. As before, cardiac parameters will be measured. In Group 3, rats will be administered with DOX (2.5 mg/kg, IV) and MitoQ (5 mg/kg) twice/week for 10-12 weeks. In Group 4, rats will be administered with MitoQ (5 mg/kg) twice/week for 10-12 weeks. Several parameters (e.g., left ventricular ejection fraction, fractional shortening, left ventricular mass and wall thickness and radial strain) will be measured in DOX-treated, tumor-bearing rats. Likewise, the effects of DOX, MitoQ and DOX plus MitoQ on tumor size and tumor volume will be monitored using histological methods.

Results (Prophetic): MitoQ treatment will ameliorate DOX-induced cardiotoxicity in tumor-bearing rats. At the same time, MitoQ will potentate DOX-induced breast cancer regression in DMBA-treated rats.

The invention has been described in connection with what are presently considered to be the most practical and preferred embodiments. However, the present invention has been presented by way of illustration and is not intended to be limited to the disclosed embodiments. Accordingly, those skilled in the art will realize that the invention is intended to encompass all modifications and alternative arrangements within the spirit and scope of die invention as set forth in the appended claims.

The invention claimed is:

1. A method for treating a subject having a cancer comprising the step of:
administering an effective amount of a mitochondria-targeted antioxidant to a subject having or susceptible to a cancer so that the cancer is reduced.

2. A method as recited in claim 1, further including co-administering an effective amount of an anthracycline.

3. A method as recited in claim 1, wherein the mitochondria-targeted antioxidant is MitoQ

4. A method as recited in claim 3, wherein MitoQ is [10-(4,5-dimethoxy-2-methyl-3,6-dioxo-1,4-cyclohexadien-1-yl)decyl]triphenylphosphonium bromide.

5. A method as recited in claim 2, wherein the anthracycline is selected from the group consisting of doxorubicin, daunorubicin, epirubicin, idartibicin and mitoxantrone.

6. A method as recited in claim 2, wherein the anthracycline is doxorubicin.

7. A method as recited in claim 1, wherein the cancer is selected from the group consisting of Hodgkin's disease, breast cancer, prostate cancer and leukemia.

8. A method for preventing anthracycline-induced toxicity in normal cells comprising the step of:
co-administering an effective amount of a mitochondria-targeted antioxidant with an effective amount of an anthracycline to a subject having or at-risk for anthracycline-induced toxicity.

9. A method as recited in claim 8, wherein the normal cells are cardiomyocytes.

10. A method as recited in claim 8, wherein the mitochondria-targeted antioxidant is MitoQ.

11. A method as recited in claim 9, wherein MitoQ is [10-(4,5-dimethoxy-2-methyl-3,6-dioxo-1,4-cyclohexadien-1-yl)decyl]triphenylphosphonium bromide.

12. A method as recited in claim 8, wherein the anthracycline is doxorubicin, daunorubicin, epirubicin, idarubicin and mitoxantrone.

13. A method as recited in claim 8, wherein the anthracycline is doxorubicin.

14. A method as recited in claim 10, wherein the subject having or at-risk for anthracycline-induced toxicity has cancer.

15. A method as recited in claim 14, wherein the cancer is selected from the group consisting of Hodgkin's disease, breast cancer, prostate cancer and leukemia.
